(19) United States
(12) Patent Application Publication
(10) Pub. No.: US 2024/0099802 A1
(43) Pub. Date: Mar. 28, 2024

(54) DEVICE FOR INCREASING FORESKIN COVERAGE OF GLANS

(71) Applicant: Tyler Mitchell-Mayeux Picard, New Iberia, LA (US)

(72) Inventor: Tyler Mitchell-Mayeux Picard, New Iberia, LA (US)

(21) Appl. No.: 17/936,046

(22) Filed: Sep. 28, 2022

(51) Int. Cl. A61B 90/00; A61F 5/41

(52) U.S. Cl. CPC A61B 90/02; A61F 5/41

(57) ABSTRACT

A restoration device for foreskin is disclosed. The device includes a base, an outer sleeve, and a compression sleeve. The base defines a glans cone designed to receive a user glans, in use. The outer sleeve includes an inner portion that matingly couples with the base and a sleeve outer ring designed to press on a user foreskin, in use. The compression sleeve is designed to wrap around an outer surface of the sleeve outer ring, in use.

### BACKGROUND OF THE INVENTION

The present invention relates to devices to increase foreskin coverage and, more particularly, to a modular, silicone device for increasing foreskin coverage of the glans, in particular, for circumcised males.

Many circumcised males are unsatisfied with the appearance or function of their penis, and current devices have several common problem areas that can be improved upon. For example, existing devices are cone-shaped, which increases stress on the skin at the bottom of the cone as it hugs a tight corner. Most other devices are made of plastic, which is harder and less comfortable. Other devices only offer one or two methods of restoration per device, forcing customers to purchase multiple devices for a well-rounded experience. Further, uncomfortable restoration sessions will be shorter and prone to possible abrasion of the skin. Many individuals cannot afford to purchase hundreds or thousands of dollars of equipment, with some opting for unsafe homemade solutions.

As can be seen, there is a need for a modular device for increasing foreskin coverage, as described herein.

### SUMMARY OF THE INVENTION

In one aspect of the present invention, a restoration device for foreskin is disclosed that includes: a base defining a glans cone configured to receive a user glans, in use; an outer sleeve comprising an inner portion that matingly couples with the base and a sleeve outer ring configured to press on a user foreskin, in use; and a compression sleeve configured to wrap around an outer surface of the sleeve outer ring, in use.

These and other features, aspects and advantages of the present invention will become better understood with reference to the following drawings, description, and claims.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 is a perspective view of an embodiment of the present invention, shown in use;

FIG. 2 is an exploded view of the embodiment of the present invention;

FIG. 3 is a section view of the embodiment of the present invention, taken along line 3-3 in FIG. 1;

FIG. 4 is a section view of a base of the embodiment of the present invention, illustrating the insertion on glans into a cone;

FIG. 5 is a section view of the embodiment of the present invention, illustrating foreskin being pulled over the sides of the base in preparation to place an outer sleeve;

FIG. 6 is a section view of the embodiment of the present invention, illustrating the pulling back of an outer ring to prepare for installation;

FIG. 7 is a section view of the embodiment of the present invention, illustrating legs being placed to capture the foreskin in a slot;

FIG. 8 is a section view of the embodiment of the present invention, illustrating the stretching of a compression sleeve to fit over the outer sleeve;

FIG. 9 is a section view of the embodiment of the present invention, with all elements in place;

FIG. 10 is a perspective view of an alternate size of the base of the embodiment of the present invention; and

FIG. 11 is a section view of the embodiment of the present invention, taken along line 11-11 in FIG. 10.

### DETAILED DESCRIPTION OF THE INVENTION

The subject disclosure is described with reference to the drawings, wherein like reference numerals are used to refer to like elements throughout. In the following description, for purposes of explanation, numerous specific details are set forth in order to provide a thorough understanding of the present disclosure such that one skilled in the art will be enabled to make and use the present invention. It may be evident, however, that the present disclosure may be practiced without some of these specific details. For the purpose of clarity, technical material that is known in the technical fields related to the present invention has not been described in detail so that the present invention is not unnecessarily obscured.

Broadly, one embodiment of the present invention is a device that holds the remnant foreskin in place and stretches it to encourage growth and increased coverage of the glans. The present invention includes a modular design and has attachments that provide various methods of tensioning skin.

The present invention utilizes a pliable material, such as skin-safe silicone, and has a unibody design that prevents twisting and is soft. Customers have the option of using various tension methods (tugging/air/weight/packing) all with one device due to its modular design. It may be formed with a cylinder-shaped design and beveled edges that relieve stress on the skin. In use, the wearer's skin is held in place by compression of soft silicone. Further, the present invention enables the wearer to urinate without removing the device, which is a functionality not currently found in this field. The present invention introduces a more comfortable experience and is more cost-effective than the prior art. This may decrease the time required for satisfactory results, decrease the risk of injury, and appeal to budget-conscious consumers.

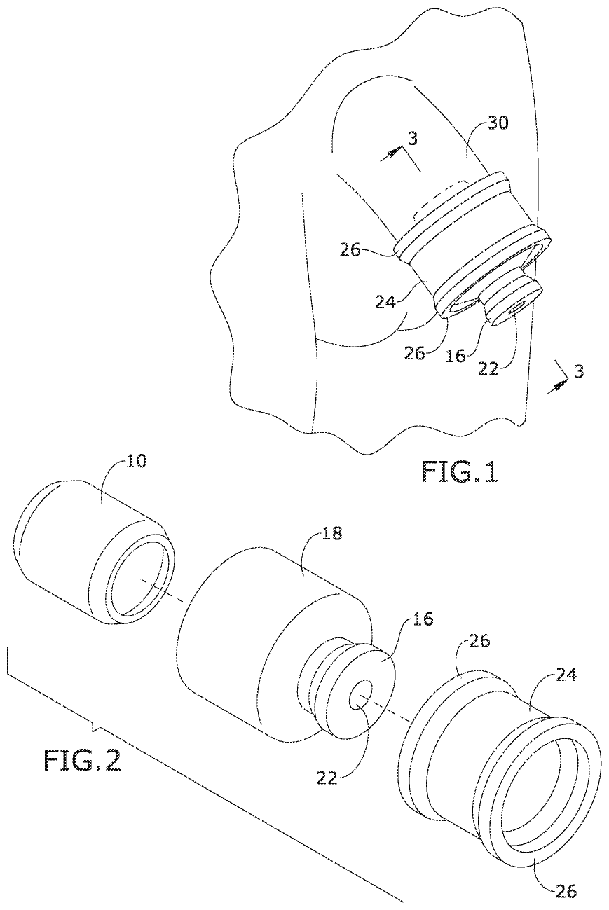

Referring now to FIGS. 1 and 2, a foreskin restoration device is generally shown. Specifically, and as shown in FIG. 2, the device may include a base 10, an outer sleeve 16, and a compression sleeve 24, which all may be generally conical in shape. These components, as described in greater detail below, in combination function to help restore the foreskin 30 of a user.

As depicted in the cross-sectional view of FIG. 4, the base 10 may include a glans cone 12 (which is funnel-shaped) for receiving a tip portion of a male glans 28. At the end of the glans cone 12 is a cone opening 14 designed for urine and air to pass through, in use.

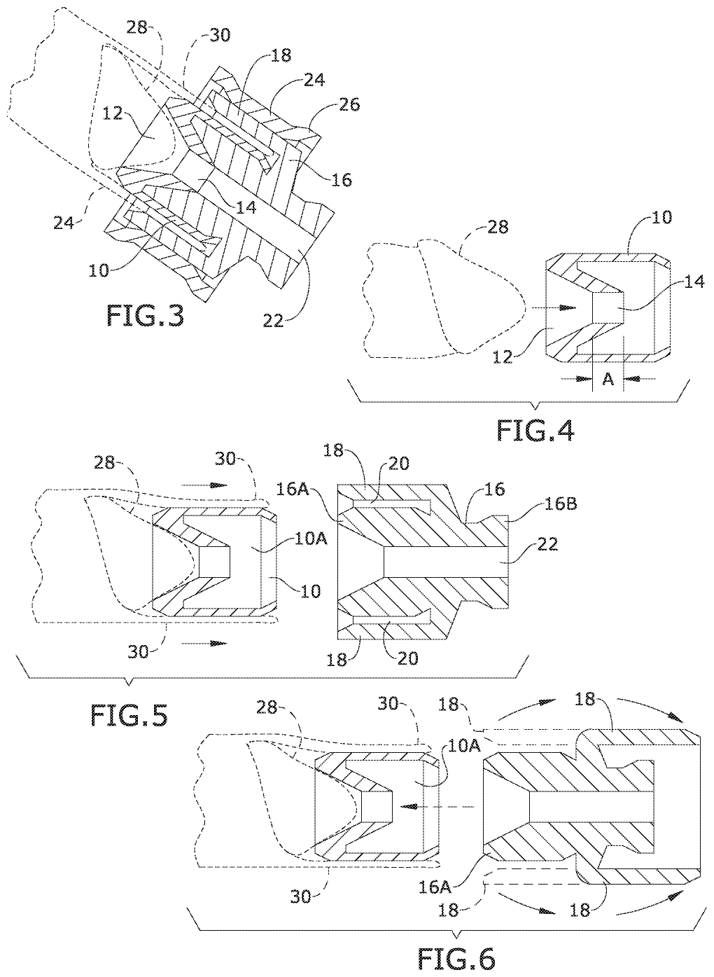

As shown in the cross-sectional view of FIG. 5, the outer sleeve 16 includes an inner portion 16A that, in use, is received in a cavity 10A of the base 10. As shown in FIG. 3, they are complementarily shaped to engage one another. A sleeve outer ring 18 forms an outermost portion of the outer sleeve 16 and surrounds the inner portion 16A. Between the sleeve outer ring 18 and the inner portion 16A is a foreskin slot 20 for receiving the foreskin 30 of the user. Like the glans cone 10, the outer sleeve 16 defines a sleeve opening 22 designed for urine and air to pass, through, in use. A distal end 16B of the outer sleeve (right side as depicted in FIG. 5) can be used to hook up various accessories.

As shown in FIG. 2, the compression sleeve 24 is provided with flared ends 26. In an unstretched state, an inner diameter of the sleeve 24 is smaller than an outer diameter of the sleeve outer ring 18. As shown in FIG. 8, the compression sleeve 24 can be stretched such that it is temporarily greater than the outer diameter of the sleeve outer ring 18.

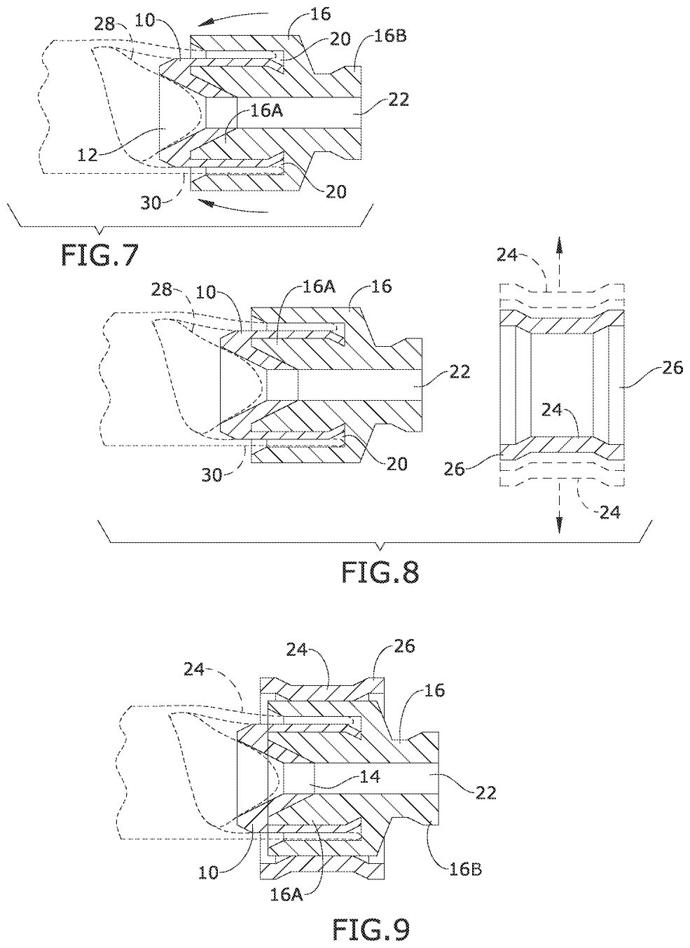

A method of using the present invention is generally depicted in the sequence from FIGS. 4-9. First, the base 10 is positioned on the tip portion of the user's glans. Next, the user's foreskin is pulled over an outer surface of the base to substantially encircle the base 10. The sleeve outer ring 18, due to its pliability, can be rolled back (see FIGS. 5-6). Then, the inner portion 16A of the outer sleeve 16 is slid to nest with the cavity 10A of the base 10. As shown in FIG. 7, the base 10 has an inwardly angled end portion that is received in an annular notch in the inner portion 16A to retain it in place. From there, and as denoted by the directional arrows in FIG. 7, the sleeve outer ring 18 is folded down to sandwich the foreskin 30 between it and the base 10. To securely clamp down on the foreskin and prevent it from sliding out of the channel defined between the base 10 and the outer sleeve 16, the compression sleeve 24 is stretched (denoted by the phantom lines in FIG. 8) and slid over the outer sleeve 16. Due to its pliable and elastic nature, it compress back down in size, thus applying a compression force to the outer surface of the outer sleeve 16. Thus, the restoration device is securely maintained in place, and the user has an opening through which they can urinate through and air can pass through.

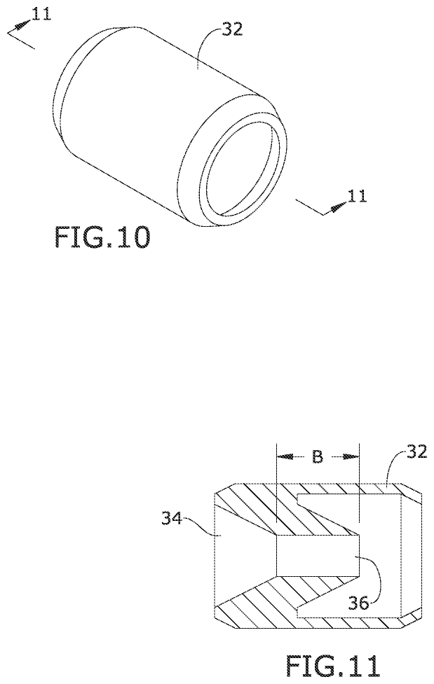

As shown in FIGS. 10-11, an additional base 32 may be provided as part of a kit for more advanced users. As shown, it has a glans cone 34 and a longer opening 36 than the other base 10. See length B in FIG. 11 compared with length A shown in FIG. 4. This allows additional room for a deeper stretching of the skin during tugging and weight use. This results in greater device stability and a more thorough and even stretch by holding the glans 28 further away from where the foreskin 30 is anchored. It also helps to stabilize the skin balloon created during air inflation.

As those with skill in the art will appreciated, multiple bases 10 may be stackable, meaning that multiple bases 10 can be used simultaneously with the outer sleeve 16 to push the glans 28 back and pull the foreskin 30 forward.

The desired result is typically to regenerate enough foreskin 30 to cover the glans 28 of the penis. Users may do this by wearing the restoration device, e.g., multiple times per day, one to three hours at a time, several days per week. Users must apply tension for the device to work properly—including air, weight, outer sleeve, or tugging. More specifically: Air: A one-way valve may be inserted into the hole 22 and air is pumped in via hand pump (like from a blood pressure cuff). Weight: a weight may be attached to the outer sleeve 16 (the tip of the device) and hung freely. Outer sleeve 16: They extend inner portion 16A to make it longer, accommodating those with more foreskin 30 and allowing for a better stretch. Tugging: A silicone/bungee cord accessory may be attached to the outer sleeve 16 (the tip of the device). The user may pull on the bungee cord or attach it to a leg strap/brace to create tension. Repeating the process over an extended period of time, e.g., several years, should encourage coverage.

In exemplary embodiments, the present invention may be formed in the following manner. Molds may be three-dimensional printed in polyethylene terephthalate glycol (PETG) filament. Multiple pieces may be necessary for molds as the shapes are more complex. Molds may be sanded smooth with a rotary tool and hot-glued together. They may also be coated in epoxy resin for a smoother, non-stick finish. Silicone may be mixed, vacuum degassed, and poured into the molds' reservoir on a vibration plate to release air and fill in every crack. The molds may then be taken apart by scraping off the hot glue, removing the top reservoir and using a wedge, chisel, or putty knife to open the mold. Remnant flashing material may be trimmed from the demolded product with scissors or craft knives. The product may then be sanded smooth where necessary with a rotary tool using a sandpaper wheel.

While one or more preferred embodiments are disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

While apparatuses and methods are described in terms of “comprising,” “containing,” or “including” various components or steps, the apparatuses and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

What is claimed is:

1. A restoration device for foreskin comprising:
a base defining a glans cone configured to receive a user glans, in use;
an outer sleeve comprising an inner portion that matingly couples with the base and a sleeve outer ring configured to press on a user foreskin, in use; and
a compression sleeve configured to wrap around an outer surface of the sleeve outer ring, in use.

2. The restoration device of claim 1, wherein the base, the outer sleeve, and the compression sleeve comprise a pliable material.

3. The restoration device of claim 1, wherein the base defines a cone opening and the outer sleeve defines an outer sleeve opening that aligns with the cone opening.

4. The restoration device of claim 1, wherein the sleeve outer ring is operable to fold between a first position, where a distal end thereof is distal the inner portion, and a second position, where the distal end thereof is proximal the inner portion.

5. The restoration device of claim 1, wherein the compression sleeve is operable to apply a compression force to the outer surface of the sleeve outer ring, in use.

6. The restoration device of claim 1, wherein a distal end of the outer sleeve is configured to connect to one of a plurality of accessories.
